(19) United States
(12) Patent Application Publication
(10) Pub. No.: US 2013/0172877 A1
(43) Pub. Date: Jul. 4, 2013

(54) BALLOON EXPANDABLE MULTI-ELECTRODE RF ABLATION CATHETER

(71) Applicant: BOSTON SCIENTIFIC SCIMED, INC., Maple Grove, MN (US)

(72) Inventors: RAJ SUBRAMANIAM, FREMONT, CA (US); ZAYA TUN, LIVERMORE, CA (US)

(73) Assignee: BOSTON SCIENTIFIC SCIMED, INC., MAPLE GROVE, MN (US)

(21) Appl. No.: 13/709,867

(22) Filed: Dec. 10, 2012

Related U.S. Application Data
(60) Provisional application No. 61/580,967, filed on Dec. 28, 2011.

(51) Int. Cl. A61B 18/00

(52) U.S. Cl. CPC A61B 18/00
USPC 606/41

(57) ABSTRACT

An intravascular catheter for nerve modulation through the wall of a blood vessel, comprising an shaft having a proximal end and a distal end and a central axis, a balloon disposed on the shaft and having a proximal end, a distal end, an interior surface, and exterior surface, a lumen defined by the interior surface, a plurality of electrodes disposed on the balloon, and a plurality of elastomeric members disposed between the plurality of electrodes and the balloon and extending between the proximal end of the balloon and the distal end of the balloon.

### CROSS-REFERENCE TO RELATED APPLICATIONS

This application claims priority under 35 U.S.C. §119 to U.S. Provisional Application Ser. No. 61/580,967, filed Dec. 28, 2011, the entirety of which is incorporated herein by reference.

### FIELD

The invention generally pertains to percutaneous and intravascular devices for nerve modulation and/or ablation.

### BACKGROUND

Certain treatments require the temporary or permanent interruption or modification of select nerve function. One example treatment is renal nerve ablation which is sometimes used to treat conditions related to congestive heart failure. The kidneys produce a sympathetic response to congestive heart failure, which, among other effects, increases the undesired retention of water and/or sodium. Ablating some of the nerves running to the kidneys may reduce or eliminate this sympathetic function, which may provide a corresponding reduction in the associated undesired symptoms.

Many nerves (and nervous tissue such as brain tissue), including renal nerves, run along the walls of or in close proximity to blood vessels and thus can be accessed intravascularly through the walls of the blood vessels. In some instances, it may be desirable to ablate perivascular nerves using a radio frequency (RF) electrode. In other instances, the perivascular nerves may be ablated by other means including application of thermal, ultrasonic, laser, microwave, and other related energy sources to the vessel wall.

Because the nerves are hard to visualize, treatment methods employing such energy sources have tended to apply the energy as a generally circumferential ring to ensure that the nerves are modulated. However, such a treatment may result in thermal injury to the vessel wall near the electrode and other undesirable side effects such as, but not limited to, blood damage, clotting, weakened vessel wall, and/or protein fouling of the electrode.

### SUMMARY

It is therefore desirable to provide for alternative systems and methods for intravascular nerve modulation which distribute ablation sites along and around the vessel.

Some embodiments of the invention are directed to a balloon catheter configured for nerve modulation and/or ablation. The balloon catheter includes an inflatable balloon at or proximate a distal end of the device. A plurality of electrodes may be disposed on the balloon. The electrodes are preferably spaced from each other circumferentially and radially. A plurality of elastomeric members may also be provided. These elastomeric members may be on the outer surface of the balloon and may be configured to help collapse the balloon to a closed profile when the balloon is deflated. In some embodiments, some of the elastomeric members may be elastomeric bands that extend generally longitudinally. The bands may be attached at a proximal end of the balloon and/or at a distal end of the balloon. The bands are preferably under tension when the balloon is inflated and are configured to be under tension even when the balloon is in a deflated condition. The elastomeric members may be disposed between the electrodes and the balloons such that one elastomeric member is disposed under each electrode. In some embodiments, one or more elastomeric members may run circumferentially around the balloon. These circumferential elastomeric members may also be disposed under the balloon. In some embodiments, flexible circuit or other suitable electrode. In some embodiments, the elastomeric members may be elastomeric tubes, and the wire to the electrode and optionally one or more sensor wires such as a thermocouple are disposed within the tube. In some embodiments, the balloon has a conventional generally cylindrical shape. In other embodiments, the balloon may be ridged or lobed or may have an acorn shape for use with an antrum of a pulmonary vein or like anatomy. The catheter system may include other features such as a steering wire and proximal knob to make the catheter bi-directionally steerable and conventional features such as a guidewire lumen configured for over-the-wire or for monorail delivery.

In one illustrative method of use, a balloon catheter according to an embodiment of the invention is inserted percutaneously and/or intravascularly to a treatment location using a guidewire, a guide catheter or other conventional means. The balloon is inflated and preferably so that the electrodes are in contact with or immediately adjacent a vessel wall. The electrode is activated and RF energy is transmitted into the adjacent tissue. The treatment may be ended after a predetermined time. The tension in the elastomeric members may serve to keep the balloon in a low profile during delivery or when deflated.

The above summary of some example embodiments is not intended to describe each disclosed embodiment or every implementation of the invention.

### BRIEF DESCRIPTION OF DRAWINGS

The invention may be more completely understood in consideration of the following detailed description of various embodiments in connection with the accompanying drawings, in which:

FIG. 1 is a schematic view illustrating a renal nerve modulation system in situ.

FIG. 2 is a schematic view illustrating the distal end of a renal nerve modulation system.

FIG. 3 is a cross-sectional side view of the renal nerve modulation system of FIG. 2 in situ.

FIG. 4A is a schematic view illustrating the distal end of a renal nerve modulation system.

FIG. 4B is a cross-sectional view illustrating the renal nerve modulation system of FIG. 4A.

FIG. 5 is a schematic view illustrating the distal end of a renal nerve modulation system.

FIG. 6 is a schematic view illustrating the distal end of a renal nerve modulation system.

FIG. 7 is a detail view of the renal nerve modulation system of FIG. 6.

While the invention is amenable to various modifications and alternative forms, specifics thereof have been shown by way of example in the drawings and will be described in detail. It should be understood, however, that the intention is not to limit aspects of the invention to the particular embodiments described. On the contrary, the intention is to cover all modifications, equivalents, and alternatives falling within the spirit and scope of the invention.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, “an”, and “the” include the plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect such feature, structure, or characteristic in connection with other embodiments whether or not explicitly described unless cleared stated to the contrary.

While the devices and methods described herein are discussed relative to renal nerve modulation through a blood vessel wall, it is contemplated that the devices and methods may be used in other applications where nerve modulation and/or ablation are desired. The term modulation refers to ablation and other techniques that may alter the function of affected nerves. When multiple ablations are desirable, they may be performed sequentially by a single ablation device mounted on an elongate member extending along a central elongate axis of the blood vessel, said elongate member having a generally helical radially self-expanding region disposed proximate the distal end wherein at least one ablation device is mounted along a generally helical portion of the elongate member.

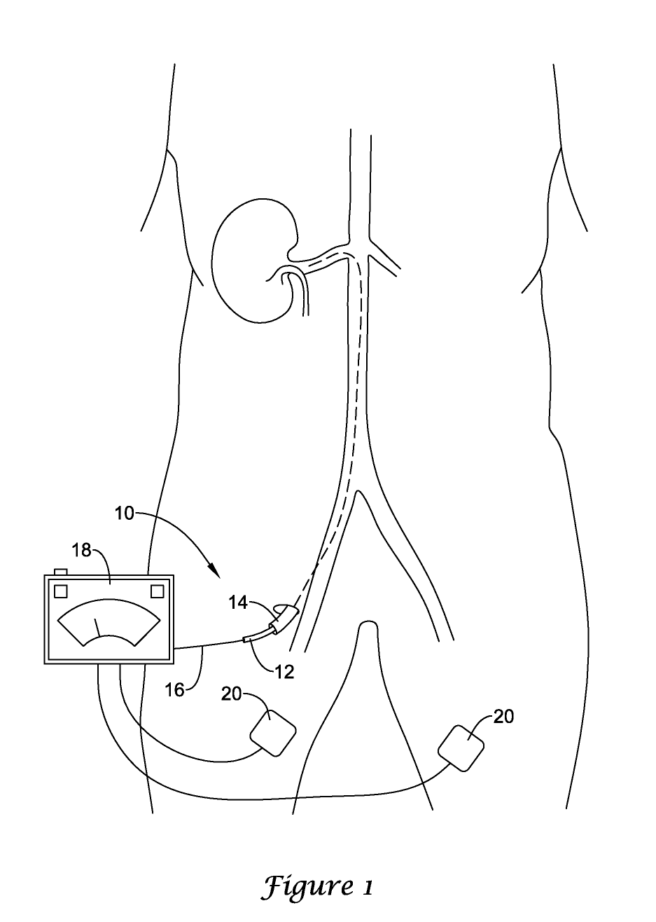

FIG. 1 is a schematic view of an illustrative renal nerve modulation system in situ. System 10 may include one or more conductive element(s) 16 for providing power to a renal ablation system including a renal nerve modulation device 12 disposed within a delivery sheath 14, which may be adapted to slidably contain the renal nerve modulation device 12 when the radially expanding region (not shown) of the elongate member is in a non-expanded configuration, the details of which can be better seen in subsequent figures. A proximal end of conductive element(s) 16 may be connected to a control and power element 18, which supplies necessary electrical energy to activate one or more electrodes to which the distal end of wire(s) 16 are attached at or near a distal end of the renal nerve modulation device 12. When suitably activated, the electrodes are capable of ablating tissue as described below. The terms electrode and electrodes may be considered to be equivalent to elements capable of ablating adjacent tissue in the disclosure which follows. Suitable materials for the delivery sheath 14, elongate member 12 and elements capable of ablating adjacent tissue are known in the art and in some embodiments may include internal and/or external layers of lubricious material(s). In some instances, return electrode patches 20 may be supplied on the legs or at another conventional location on the patient's body to complete the circuit. A proximal hub (not illustrated) having ports for a guidewire, an inflation lumen and a return lumen may also be included.

The control and power element 18 may include monitoring elements to monitor parameters such as power, temperature, voltage, pulse size and/or shape and other suitable parameters, with sensors mounted along renal nerve modulation device 12, as well as suitable controls for performing the desired procedure. In some embodiments, the power element 18 may control a radio frequency (RF) electrode. The electrode may be configured to operate at a frequency of approximately 460 kHz. It is contemplated that any desired frequency in the RF range may be used, for example, from 450-500 kHz. It is further contemplated that other ablation devices may be used as desired, for example, but not limited to resistance heating, ultrasound, microwave, and laser devices and these devices may require that power be supplied by the power element 18 in a different form. The control and power element 18 may be attached to the one or more electrodes in a manner as to allow control of each of the electrodes independently from the others.

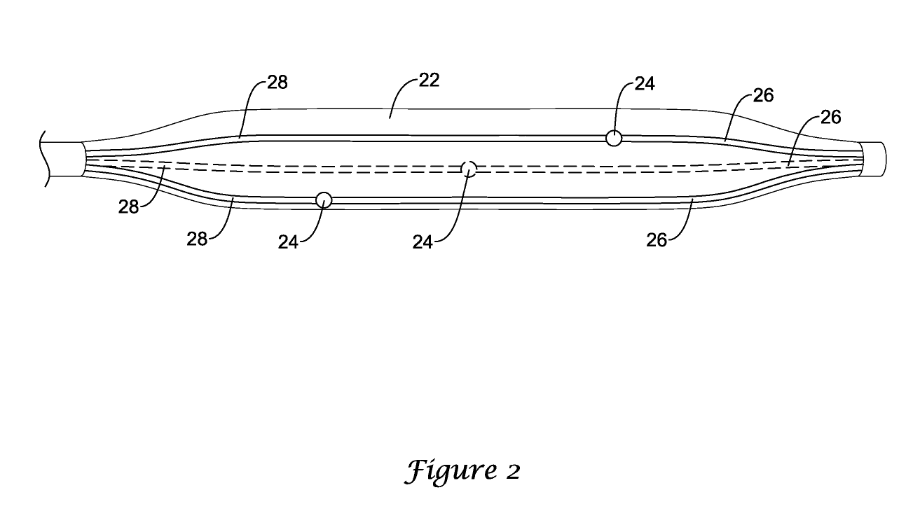

FIG. 2 illustrates the distal portion of a renal nerve modulation device 12. Renal nerve modulation device 12 includes a balloon 22 having at least one electrode 24 disposed on an outer surface thereof. In the FIG. 2 embodiment, three electrodes 24 are illustrated. Embodiments are contemplated that include various numbers of electrodes, such as 1, 2, 3, 4, 5, 6 or more electrodes. In some embodiments, the electrodes are spaced longitudinally and circumferentially as illustrated. In other embodiments, the electrodes 24 may be at the same axial location and may also be spaced about the circumference of the balloon. A conductor 28 may extend proximally from each electrode 24 to electrically connect the electrodes through conductive element(s) 16 to the control and power element 18. One or more sensors (not illustrated) such as thermocouples may also be disposed on the balloon proximate the one or more electrodes 24 and connected to the control and power element 18.

The catheter system also includes elastomeric members 26. Elastomeric member 26 may be elastomeric tension members and may have a flat ribbon profile. In the FIG. 2 embodiment, an elastomeric member runs under each of the electrodes 26. The elastomeric members 26 may extend from the electrodes 24 to the distal end of the balloon 22 or may extend from the proximal end of the balloon 22 to the distal end of the balloon. The elastomeric members 26 keep the electrodes 24 in a low profile when the balloon is not inflated and help to collapse the electrodes to a low profile when the balloon is deflated.

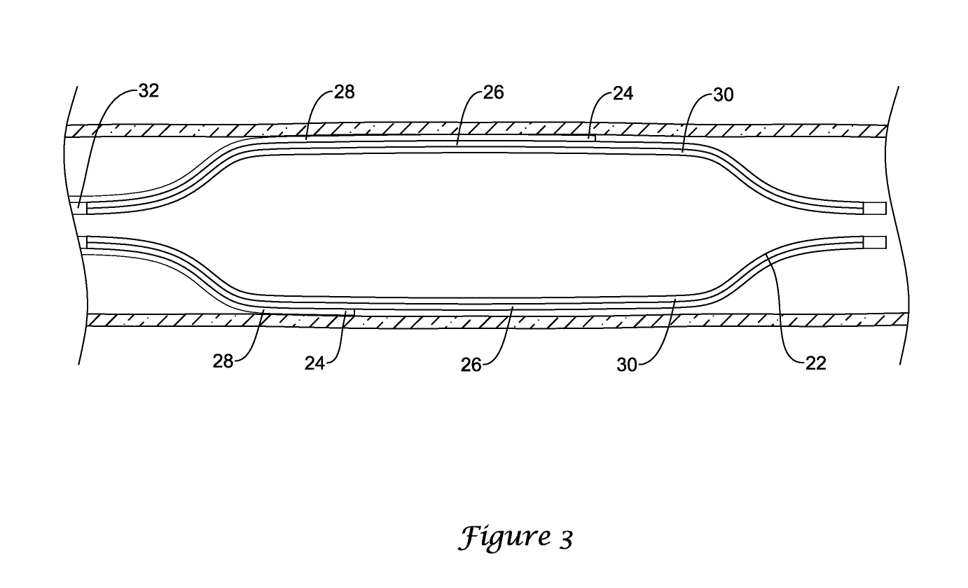

As illustrated in FIG. 3, when the balloon 22 is inflated, the electrodes 24 are in contact with the tissue of the vessel wall. A shaft 32 is attached to the balloon. Shaft 32 may include an inflation lumen, a guide wire lumen and other lumens as is conventional and may be attached to the balloon 22 at the proximal end of the balloon 22 or may extend through the lumen of the balloon 22 and be attached to the balloon at both the proximal and distal ends of the balloon. As illustrated, the elastomeric member 26 runs from the proximal end of the balloon 22 to the distal end of the balloon 22 over the balloon wall 30 and under the electrodes 24.

The electrodes 24 and conductors 28 may be made as a flexible circuit. A flexible circuit generally has a conductive layer sandwiched by two dielectric layers. In the case of an electrode 24 and conductor 28, the conductor portion would include the conductive layer and both dielectric layers while the electrode portion would include the conductive layer and only the bottom dielectric layer. In other embodiments, the electrodes 24 may be formed directly on the surface of the balloon 22 or formed separately and attached to the balloon. For example, the electrodes 24 may be plated, printed, or otherwise deposited on the surface. In some instances, the electrodes 24 may be radiopaque. The electrodes 24 may be formed from any suitable material such as, but not limited to, platinum, gold, stainless steel, cobalt alloys, or other non-oxidizing materials. In some instances, titanium, tantalum, or tungsten may be used. It is contemplated that the electrodes 22 may take any shape desired, such as, but not limited to, square, rectangular, circular, oblong, etc. In some embodiments, the electrode(s) 40 may have rounded edges in order to reduce the affects of sharp edges on current density. In some instances, the electrodes may have an aspect ratio (width to length) of 1:2 or 2:1.

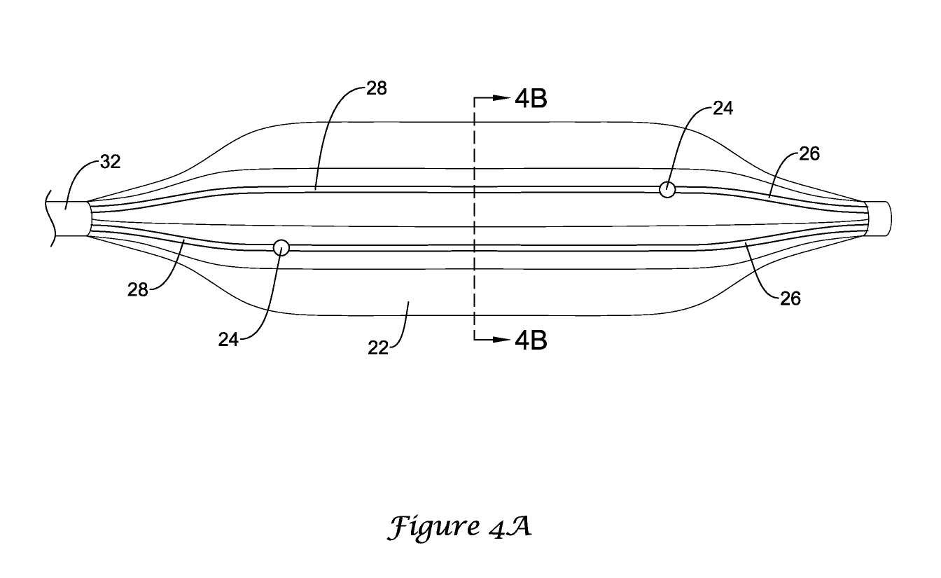

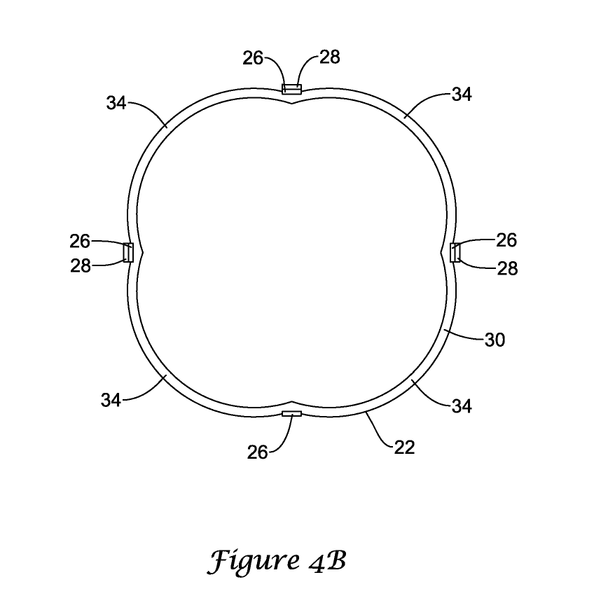

FIGS. 4A and 4B are directed to an embodiment where the balloon 22 has a ridged or lobed configuration. Each electrode 24 and the corresponding elastomeric member 26 and conductor 28 may be situated in the valley or crease between each ridge or lobe. As illustrated in FIG. 4B, in this embodiment, the balloon wall 30 is formed into four lobes 34 and each lobe 34 separates the elastomeric members 26. The balloon 22 may be molded to have such a shape or the tension in the elastomeric members 26 may force the balloon into such a shape when the balloon is inflated.

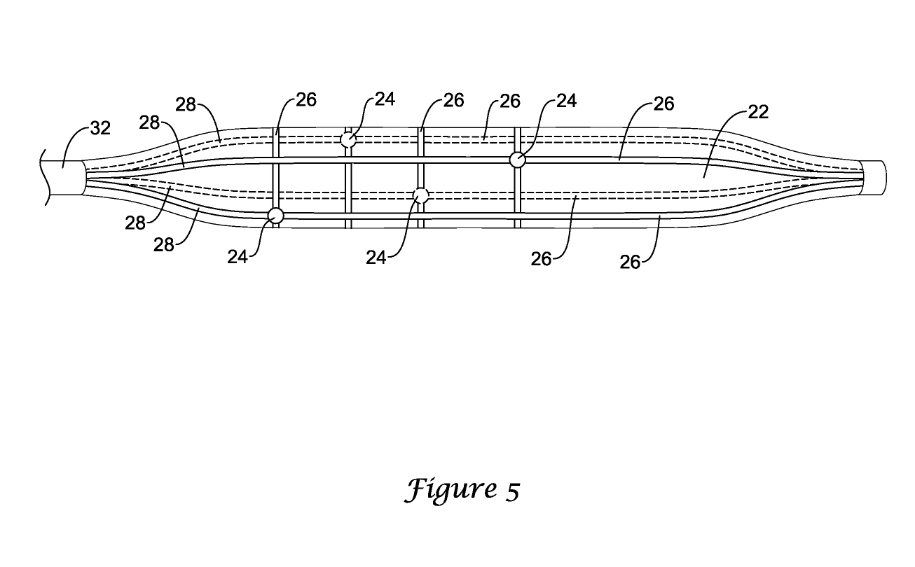

FIG. 5 illustrates an embodiment that includes circumferential elastomeric members 26 as well as the generally longitudinal elastomeric members discussed in reference to the FIG. 2 embodiment. One or more circumferential elastomeric members 26 may be disposed around the outside of the balloon. The circumferentially and generally longitudinal elastomeric members may be fixed to each other where they cross. There may be a circumferential elastomeric member 26 associated with each electrode 24 and each associated circumferential elastomeric member 26 may be disposed on the balloon wall 30 under the electrode 24. In some embodiments, fewer or more circumferential elastomeric members 26 are present. For example, in one example embodiment, a single circumferential elastomeric member 26 is disposed around the center of the balloon. In another example embodiments a first circumferential elastomeric member 26 is disposed on the balloon 22 proximal to the electrodes 24 and a second circumferential elastomeric member 26 is disposed on the balloon 22 distal to the electrodes 24.

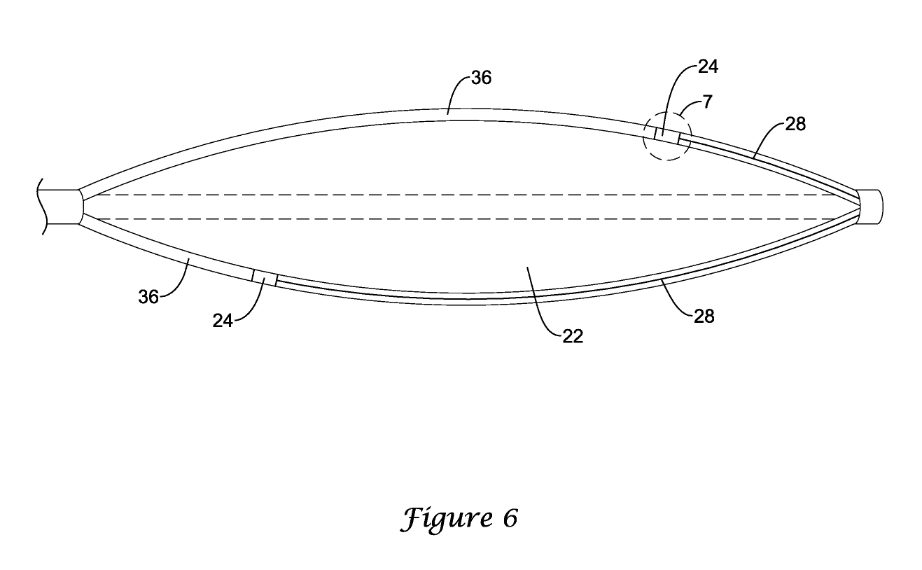

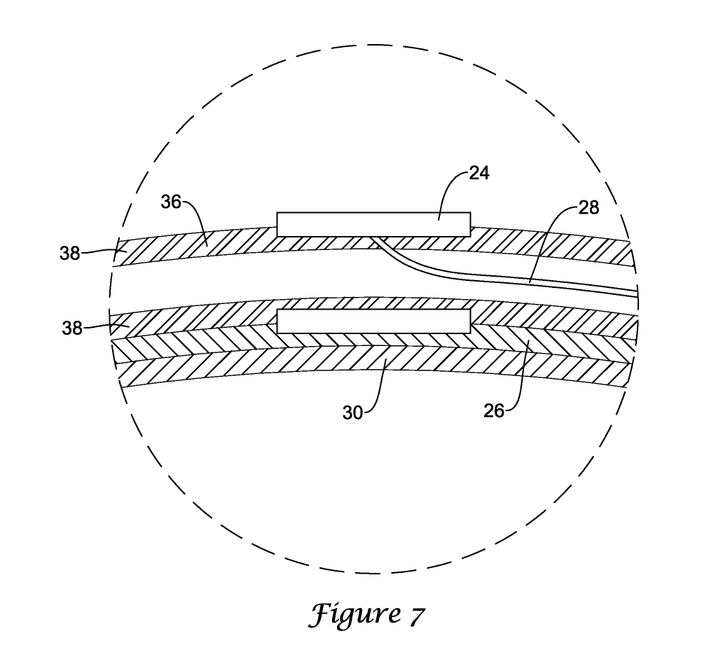

FIG. 6 illustrates an embodiment where the elastomeric members 26 may include a tube 38. An electrode 24 is disposed on the tube and may be an annular electrode disposed around the tube. The conductor 28 and any sensor elements (not illustrated) may run through the tube. FIG. 7 is a detail view illustrating the tube assembly 36. The tube assembly includes tube 38 around which electrode 24 may be disposed. Conductor 28 may run through the tube and is electrically connected to the electrode 24. The tube 38 may extend from the proximal end of the balloon to the distal end of the balloon. In some embodiments, the tube 38 is the elastomeric member and may be formed from an elastomeric material such as silicon. In other embodiments, an elastomeric member 26 is disposed between the tube 38 and the balloon wall 30. In these embodiments, tension may be provided by the elastomeric member 26 or by a combination of the elastomeric member 26 and the tube 38.

In use, a renal ablation system such as system 12 may be introduced percutaneously as is conventional in the intravascular medical device arts. For example, a guide wire may be introduced percutaneously through a femoral artery and navigated to a renal artery using standard radiographic techniques. In some embodiments, a delivery sheath 14 may be introduced over the guide wire and the guide wire may be withdrawn, and the system 12 may be then introduced through the delivery sheath. In other embodiments, the system 12 may be introduced over the guidewire, or the system, including a delivery sheath 14 may be introduced over a guidewire. In embodiments involved a delivery sheath 14, the system 12 may be delivered distally from the distal end of the delivery sheath 14 into position, or the delivery sheath may be withdrawn proximally to expose the system 12. The balloon 22 is inflated to overcome the tension in the elastomeric members 26 and expand the balloon. The balloon expansion may be monitored indirectly by monitoring the volume of fluid introduced into the system or may be monitored through radiographic or other conventional means. The electrodes 24 are then activated by supplying energy to the electrode. The energy may be supplied at 400-500 Hz and at between 0.5 and 1 amp. In some embodiments, selected electrodes are activated and deactivated to create various ablation or modulation patterns for effective therapy. The electrode 24 is preferably activated for an effective length of time, such as 1 minute or 2 minutes. One the procedure is finished at a particular location, the balloon 22 may be partially or wholly deflated and moved to a different location such as the other renal artery, and the procedure may be repeated at another location as desired using conventional delivery and repositioning techniques.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

What is claimed is:

1. An intravascular catheter for nerve modulation through the wall of a blood vessel, comprising:
an shaft having a proximal end and a distal end and a central axis;
a balloon disposed on the shaft and having a proximal end, a distal end, an interior surface, and exterior surface, a lumen defined by the interior surface;
a first electrode disposed on the balloon; and
a first elongate elastomeric member disposed between the first electrode and the balloon.

2. The catheter of claim 1 wherein the first elongate elastomeric member extends parallel to the central axis.

3. The catheter of claim 1 wherein the first elongate elastomeric member is attached to the proximal end of the balloon.

4. The catheter of claim 1 wherein the first elongate elastomeric member is attached to the distal end of the balloon.

5. The catheter of claim 1 wherein the balloon is attached to the shaft at the proximal end and the distal end.

6. The catheter of claim 1 comprising a second electrode disposed on the balloon and a second elongate elastomeric member disposed between the first electrode and the balloon.

7. The catheter of claim 1 comprising a third electrode disposed on the balloon and a third elongate elastomeric member disposed between the first electrode and the balloon.

8. The catheter of claim 6 wherein the electrodes are at different longitudinal and circumferential positions on the balloon.

9. The catheter of claim 1 further comprising a radial elastomeric member circumferentially surrounding the balloon.

10. The catheter of claim 9 wherein the radial elastomeric member is disposed under the first electrode.

11. The catheter of claim 1 wherein the electrode is a flexible circuit.

12. The catheter of claim 1 wherein the electrode is an annular electrode.

13. The catheter of claim 12 wherein the elastomeric member is a tube and the electrode is disposed around the tube.

14. The catheter of claim 1 wherein the first elastomeric member is under tension when the balloon is in an inflated state.

15. The catheter of claim 1 wherein the first elastomeric member is under tension when the balloon is in a deflated state.

16. An intravascular catheter for nerve modulation through the wall of a blood vessel, comprising:
an shaft having a proximal end and a distal end and a central axis;
a balloon disposed on the shaft and having a proximal end, a distal end, an interior surface, and exterior surface, a lumen defined by the interior surface;
a plurality of electrodes disposed on the balloon; and
a plurality of elastomeric members disposed between the plurality of electrodes and the balloon and extending between the proximal end of the balloon and the distal end of the balloon.

17. The catheter of claim 16 wherein the balloon has a plurality of elongate ridges and the plurality of electrodes are disposed between the plurality of ridges.

18. The catheter of claim 17 further comprising a control and power system attached to the electrodes and configured to independently power each of the plurality of electrodes.

19. The catheter of claim 16 wherein the plurality of elastomeric members are elastomeric tension bands having a flat ribbon profile.

20. A method of nerve modulation, comprising:
providing a catheter according to claim 1;
moving the balloon to a region of interest;
inflating the balloon; and
activating the electrode.
